(19) United States
(12) Patent Application Publication
(10) Pub. No.: US 2018/0317979 A1
(43) Pub. Date: Nov. 8, 2018

(54) Pedicle Breach Detection Device

(71) Applicant: University of Rochester, Rochester, NY (US)

(72) Inventors: Gregory Thomas Gdowski, Rochester, NY (US); Martin Gene Gira, Hilton, NY (US); Kerry Margaret Donnelly, Pittsford, NY (US); Brittany Blair Garrison, Park City, UT (US); Spencer Lee DeCinque, Little Valley, NY (US); Saadedine Anwar El-Homsi, Avon Lake, OH (US)

(73) Assignee: University of Rochester, Rochester, NY (US)

(21) Appl. No.: 15/970,643

(22) Filed: May 3, 2018

Related U.S. Application Data
(60) Provisional application No. 62/501,650, filed on May 4, 2017.

(51) Int. Cl. A61B 17/70; A61B 90/00

(52) U.S. Cl. CPC A61B 17/7092; A61B 2017/00075; A61B 90/06

(57) ABSTRACT

A pedicle breach detection device is disclosed. When a pilot hole is created during spinal fusion surgery to facilitate placement of a pedicle screw, it is important to determine if the created hole or tract has breached the vertebrae. A breach can be dangerous to the patient and cause pain, permanent damage, and the necessity for revision surgery. The present invention uses an accelerometer based shaft and ball tip to capture low level mechanical vibrations from the shaft to provide a definitive breach alert to the surgeon using audible, visual, tactile and haptic alert de vices.

### CROSS REFERENCE TO RELATED PATENT APPLICATIONS

This application claims priority to U.S. Patent Application Ser. No. 62/501,650 filed May 4 2017 entitled. “Pedicle Breach Detection Device”, the entire disclosure of which is incorporated, herein by reference.

### BACKGROUND OF THE INVENTION

### 1. Field of the Invention

This invention relates generally to advanced sensing devices, and more specifically to an advanced sensing device for detection of anomalies in a surgically created opening such as a surgical hole or tunnel in the pedicle tract of a vertebrae made during spinal surgery ,

### 2. Description of related, art

Spinal surgery, specifically spinal fusion surgery, is often performed to treat conditions of spinal instability and deformation. Spinal stenosis, degenerative disc disease, and scoliosis are examples of conditions that often require spinal fusion surgery where pedicle screws are placed in vertebrae and joined together with mechanical couplings and rods. During spinal fusion surgery, it is critically important that the pedicle screws are properly implanted to avoid breach of tile screw outside of its intended placement and outside of the cortical wall of a bone tract. Screw misplacement is currently the most common complication and leading cause of revision spinal fusion procedures. Current revision rates average 20%, and can lead to serious patient injury.

Prior to implantation of each pedicle screw, the surgeon creates a pilot hole in the vertebrae at a short bony segment of the vertebrae called the pedicle. Once a surgeon creates the pedicle tract or hole using a pedicle probe, bone probe, or similar manual drilling tool the surgeon uses a device known as a ball tip probe to confirm that there is no breach of the pedicle, tract just created. The ball tip probe is a long thin shaft with a small ball at the end, and is a simple surgical tool. In use, the ball tip probe is inserted into the created, pedicle tract, and moved around the created tract to “feel” for possible breaches. The surgeon, who must be skilled at interpreting the vibrations from the ball tip probe, determines by tactile interaction with the ball tip probe if there are any breaches in the tract. This procedure requires a significant amount of training to interpret is inherently subjective, and prone to false positives. False positives result from uncertainty in interpreting the vibrations received from the probe, and lead to unnecessary adjustments in tract creation that add time to the procedure and can lead, to lower implant stability. In addition to use of the ball tip probe after tract creation, the tract is tapped to receive the pedicle screw, and the process of feeling for a breach is repeated after tapping is complete, resulting in another opportunity for false breach determination. While the ball tip probe has been in use for many years, it requires a great deal of training and experience to use properly, and even with the most trained and experienced surgeons, is prone to error.

What is therefore needed is an advanced sensing device that eliminates the uncertainty of the ball tip probe device. What is further needed is an advanced, sensing device that provides a surgeon with positive confirmation of a breach without relying on tactile sensing of low level mechanical vibrations. What is also needed is an advanced sensing device that provides multiple modes of alert. What is further needed is an advanced sensing device that reduces surgical time, revision procedures, and complications of spinal fusion surgery.

### BRIEF SUMMARY OF THE INVENTION

In accordance with the present invention, there is provided a pedicle breach detection device comprising a slender shaft having a surgical end and an operative end; a ball tip connected, to the surgical end of the slender shall; a surgical handle connected to the operative end of the slender shaft; an accelerometer mechanically coupled to the slender shaft; and a surgical alert component configured to activate upon detection of a signal from the accelerometer that exceeds a threshold value.

The foregoing paragraph has been provided by way of introduction, and is not intended to limit the scope of the invention as described in this specification, claims and the attached drawings.

### BRIEF DESCRIPTION OF THE DRAWINGS

The invention will be described by reference to the following drawings, in which like numerals refer to like elements, and in which;

FIG. 1 is a perspective view of a pedicle breach detection device of the present invention;

FIG 2 is a perspective view of a second embodiment of the pedicle breach detection device of the present invention;

FIG. 3 is a side plan view of the pedicle breach detection device;

FIG. 4 is a top plan view of the pedicle breach detection device;

FIG. 5 is a cross sectional view of the pedicel broach detection device taken along line A-A of FIG. 4;

FIG. 6 is an alternate side plan view of the pedicle breach detection device;

FIG. 7 is a bottom plan view of the -pedicle breach detection device;

FIG. 8 is a surgical end view of the pedicle breach detection de vice;

FIG. 9 is an operative end view of the pedicle breach detection device;

FIG. 10 is a graph depicting output signals from the device and a vector summation signal of the three device output signals;

FIG. 11 is a block diagram of the electronics of the present invention; and

FIG. 12 is a flowchart depleting a computer program of the present invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and the attached drawings.

### DESCRIPTION OF THE PREFERRED EMBODIMENTS

For a general understanding of the present invention, reference is made to the drawings. In the drawings, like reference numerals have been used throughout to designate identical elements.

The present invention will be described by way of example, and not limitation. Modifications, improvements and additions to the invention described herein may be determined after reading this specification and viewing the accompanying drawings; such modifications, improvements, and additions being considered included in the spirit and broad scope of the present invention and its various embodiments described or envisioned herein.

Described and depicted herein is a pedicle breach detection device. While such a device may be useful for detecting breach in a pedicle tract during posterior instrumented spinal fusion surgeries, it may also be used to detect breaches, abnormalities or features in other surgical procedures, and the disclosure provided herein is not to be considered limiting to the provided application of pedicle tract breach detection.

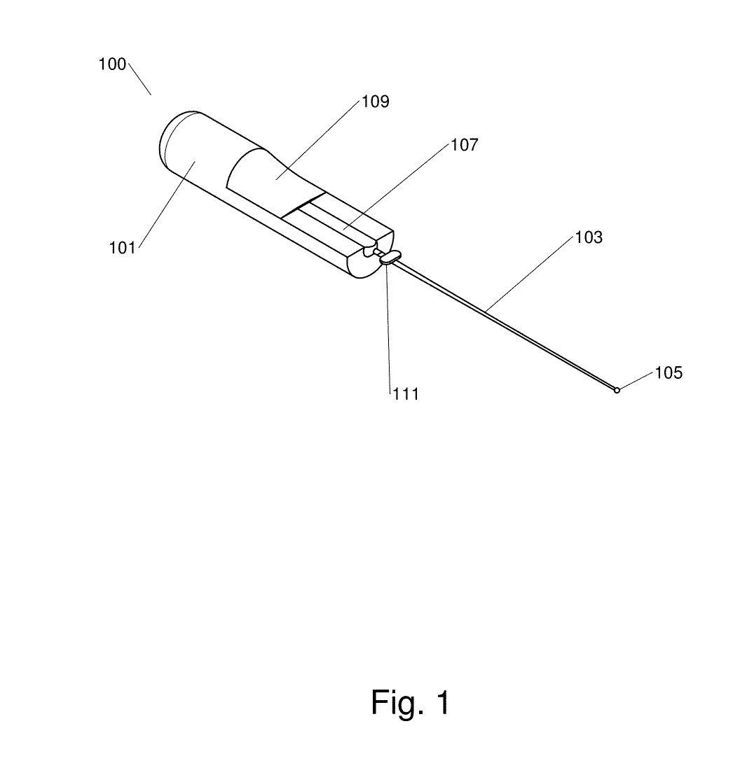

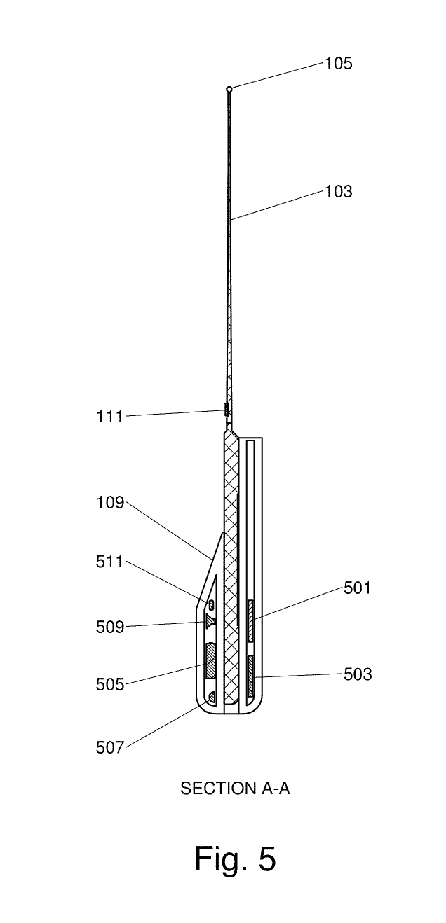

Turning first to FIG. 1, a perspective view of a pedicle breach detection device of the present invention 100 can be seen. A slender shaft 103 can be seen having a surgical end and an operative end. The operative end is connected to a surgical handle 101 and the surgical end is connected to a ball tip 105. The slender shaft 103 may be made from a variety of surgically acceptable materials, such as, for example, a stainless steel. The slender shaft 103 may be of a uniform diameter, or may, in some embodiments, have a varying diameter such that, for example, the diameter decreases toward the ball tip 105. The slender shaft 103 is needle-like to allow not only efficient surgical entry, but also to transmit vibrations from the ball tip down the length of the shaft to be received, and detected by the accelerometer or sensor 111. The shaft 103 may be rounded, square, or of a profile suitable for the transmission of vibrations from the ball tip 105 down the length of the shaft 103 toward the handle 101 where the vibrations are picked up by an accelerometer 111 or another such suitable sensor (for example, a strain gauge). In some embodiments of the present invention, a three axis accelerometer is employed. The shaft 103 may also be hollow, wound with a peripheral wire, or made from composite materials to provide efficient transmission of vibrations to the accelerometer 111. The ball tip 105 may also be made from a surgically acceptable material such as, for example, a stainless steel. The ball tip 105 may be a small spherical end, or have other geometries that are useful for probing a surgical pathway such as in spinal reconstruction or spinal fusion surgery. A shaft termination structure 107 may also be seen retained by or otherwise embedded or partially embedded in the handle 101. The shaft termination structure 107 retains the operative end of the slender shaft 103 and may also be made from a surgically acceptable material such as, for example, a stainless steel. The shaft termination, structure 107, in addition to providing an anchor element for the shaft 103, also may serve to resonate, dampen, or otherwise change the characteristics of the vibrations received through the ball tip 105 and shaft 103. The shaft termination structure may be made from a metal such as a stainless steel, or may be made from a plastic. The shaft termination structure may also be hollow or partially hollow to facilitate resonance or transmission of vibrations. In some embodiments of the present invention, the shaft 103 can be removed from the shaft termination structure 107 and associated handle 101 by way of a threaded coupling, quick release coupling, push fit coupling, or the like. The surgical handle 101 provides not only a grip for the surgeon, it also contains necessary electronics, as will be further seen and described by way of FIG. 5. The handle 101 may be made from a surgically acceptable material such as a metal, a plastic or the like. The transmission of vibrations along the shaft 103 may be picked up and modified by way of a tactile surface 109 that may be a plate or similar structure that is in acoustic communication with the shaft termination structure 107 or the shaft 103. The tactile surface 109 may act as a waveguide or resonant surface to allow a surgeon to feel the vibrations of the moving slender shaft 103 and ball tip 105 arrangement during a surgical procedure. The tactile surface 109 may also be acoustically and mechanically coupled to a haptic output 511 (see FIG. 5) that comprises an amplified vibratory signal from the shaft and ball tip with a vibratory output from a rotating motor, piezoelectric transducer, or the like. This amplified arrangement may be employed in some embodiments of the present invention, and provides a way for the surgeon to feel the construct of a surgical pathway. A sensor such as the accelerometer 111 can be seen mechanically coupled to the shaft 103 such that changes in velocity along the three axis of the shaft 103 can be detected and output as a signal that can be further processed and digitized. Placement of the sensor such as the accelerometer 111 may, in some embodiments of the present invention, be more distal from the handle, close to the ball tip 105, integrated with the shaft or integrated with or otherwise contained by or coupled to the ball tip 105. Placement of the sensor may be dictated by various design considerations, such as (but not limited to) shaft length, ball tip geometry, sensor type, and the like.

In some embodiments of the present invention, a second accelerometer or similar sensor may be incorporated elsewhere in the Pedicle Breach Detection Device, such as in the surgical handle 101 or otherwise in on attached to the distal end of the Pedicle Breach Detection Device. This second accelerometer or sensor is a differential accelerometer or sensor of sorts, the purpose of which is to sense noise and other extraneous and unwanted vibrations, and through either digital or analog means, subtract the unwanted noise signals received by the differential accelerometer or sensor from the vibrations detected by the first or primary accelerometer or sensor. Such rejection of noise signals can he implemented through, for example, the differential inputs that are found with operational amplifiers (op-amps), such as, for example, the LM741 op-amp -manufactured by Texas Instruments. The signals from both accelerometers or sensors can also be processed digitally, where the A/D converter(s) 501 (see FIG. 5) converts the analog outputs from the accelerometers or sensors into digital signals that can be further processed with a digital signal processor and or microprocessor 503 to subtract the unwanted signals from the important signals that are indicative of pedicle or surgical integrity and breach.

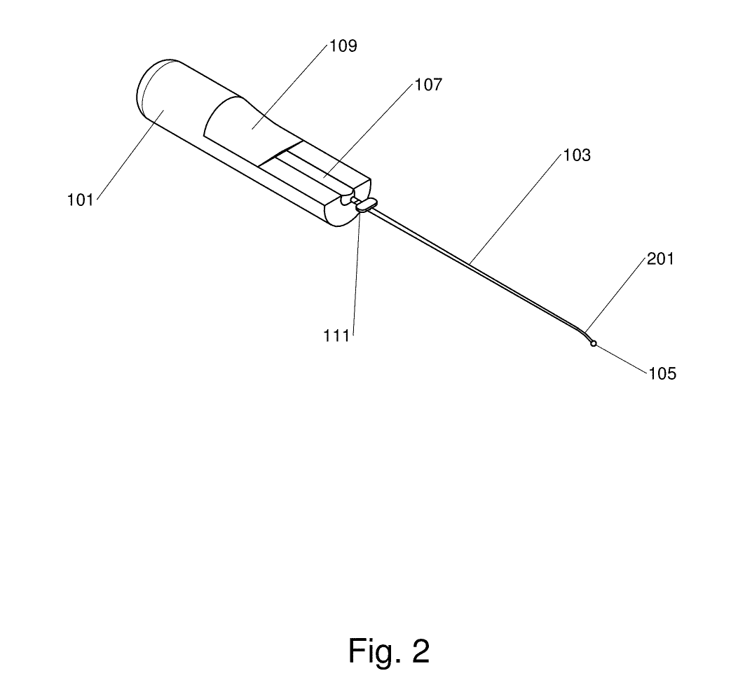

FIG. 2 is a perspective view of a second embodiment of the pedicle breach detection device of the present invention. In Fig 2, the shaft 103 has a bond or an angle to allow the surgeon to circumferentially and longitudinally probe a hole such as the pedicle tract without changing the angle at which the device of the present invention is held. This shaft angle 201 can be placed close to the ball tip 105, or set back from the ball tip.

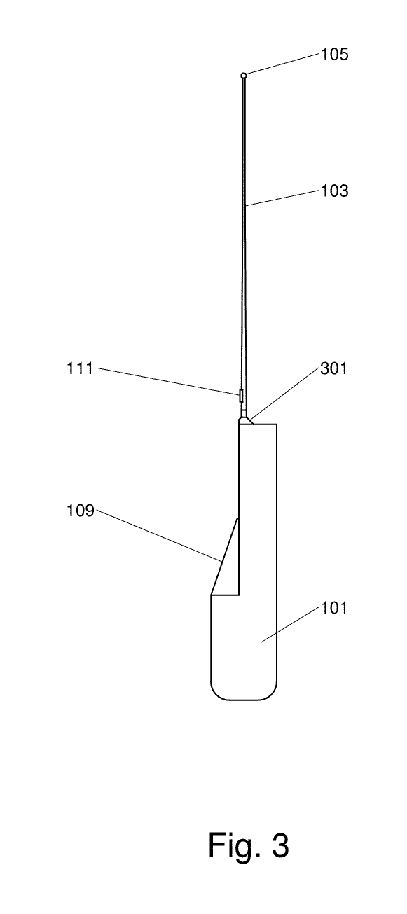

FIG. 3 is a side plan view of the pedicle breach detection device. In FIG. 3, a transition taper 301 can be seen between the slender shaft 103 and the surgical handle 101. This transition taper 301 provides structural attachment of the shaft 103 with the handle 101, and may also provide proper termination, resonance, or attenuation of vibrations along the shaft 103 while in use. The transition taper 301 may be a flare, a progressive reduction in diameter, or the like. Further, in some embodiments, the transition taper 301 may be a separate component such as a bushing, an o-ring, a spacer, or the like. The transition taper 301 may be made from a metal, a plastic, an elastomeric material, or the like.

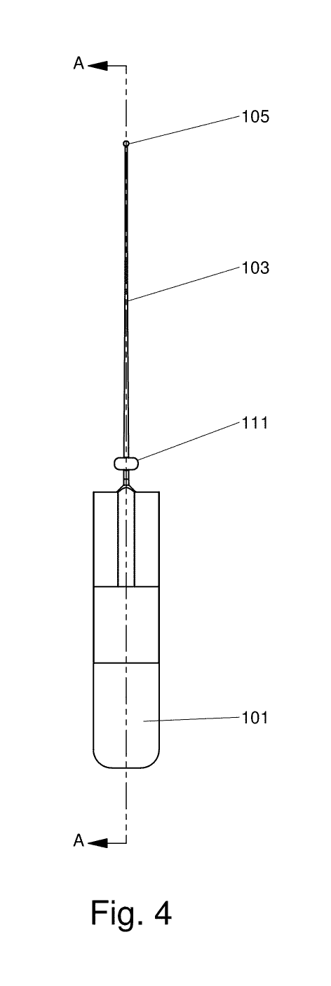

FIG. 4 is a top plan view of the pedicle breach detection device. It is important to note that while the accelerometer 111 is depicted as attached to the shaft 103 separately from the surgical handle 101, the accelerometer 111 may also be attached to the shaft 103 with the handle surrounding or otherwise retaining or encompassing the accelerometer 111. Additionally, a break or coupling can be seen between the accelerometer 111 and the surgical handle 101 which allows the shaft and ball tip to be removed from the handle for sterilization, disposal, or interchangeability with other shaft and ball tip arrangements that may be better suited to the procedure or surgeon preference. In some embodiments of the present invention, this break or coupling may be placed after the accelerometer, in other words between the ball tip 105 and the accelerometer 111. This arrangement would allow the ball tip and shaft assembly to be interchanged or replaced while maintaining the accelerometer 111 in place.

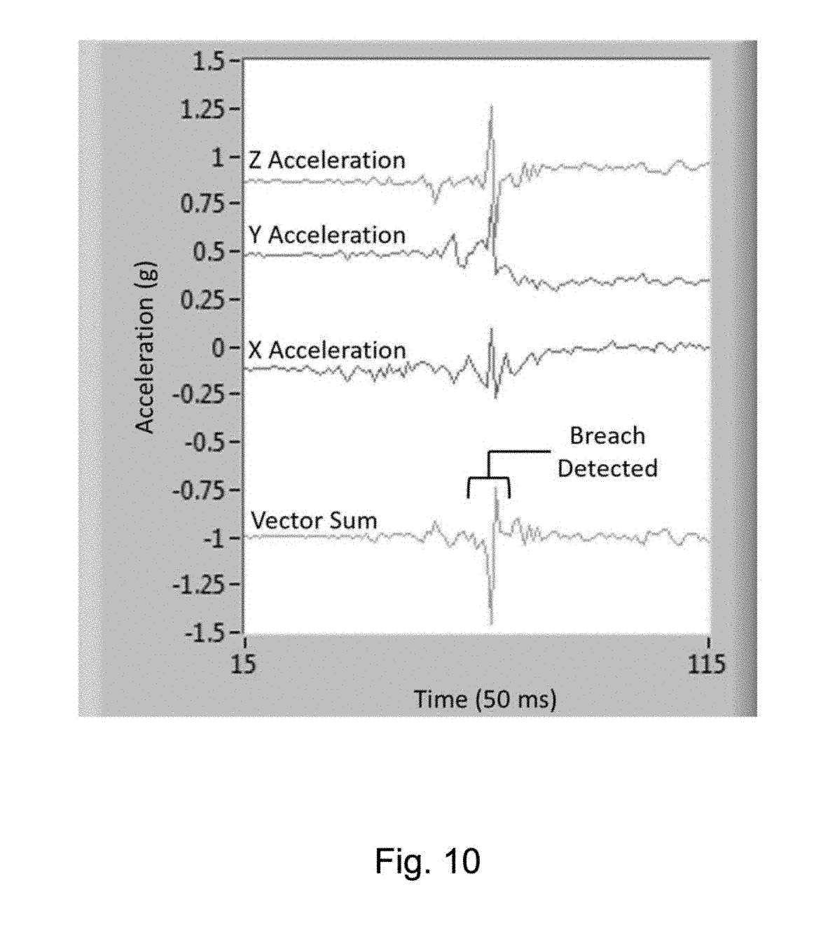

To accommodate the electronics of the Pedicle Breach Detection Device of the present invention, the surgical handle 101 may be hollow or otherwise have cavities and supporting structures to secure the necessary electronics. FIG. 5 is a cross sectional view of the pedicle breach detection device taken along line A-A of FIG. 4. The accelerometer 111 may have one or more outputs, where each output represents a spatially defined axis. In the example provided herein and depicted in FIG. 10, the accelerometer provides three axis outputs, the vector summation of which is indicative of a breach. Use of three accelerometer signals and their resulting vector summation makes the breach detection of the present invention independent of the orientation of the device by the surgeon. The accelerometer outputs are analog signals unless an accelerometer package is used that contains the requisite analog to digital converter circuitry. Otherwise, an analog to digital (A/D) converter 501 is used to convert the accelerometer outputs into digital signals that can be further processed by way of a microprocessor 503, microcontroller, computer, computing device, or computer system. In some embodiments of the present invention, the digital signals are stored and further processed by an external -computer system. Interfaces between the pedicle breach detection device and an external system or device may include both wired and wireless connections, and may also be optical or employ optics or fiber optic components. A battery or power source 505 is included, and serves to provide electrical power to the electronic components described herein. In addition to a battery, the power source may also be an ultracapacitor or an energy harvesting device. The power source 505 may be rechargeable.

Once the output signals from the accelerometer 111 are summed as a vector summation or otherwise processed in order to create an output that is indicative of a breach, that output is then compared with a threshold value that may be pre-programmed or user defined. If the threshold value is exceeded, it is indicative of a breach. Thus, if the output from the accelerometer or the processed output from the accelerometer exceeds the threshold value, a signal (a state change if the signal is digital which in turn drives the necessary surgical alert component, or an analog driving signal that directly activates the surgical alert component) is provided to a surgical alert component that activates the surgical alert component to alert the surgeon of a breach. Surgical alert components include, for example, an optical component, an audible component, a haptic component and a tactile interface or tactile surface component. Examples of such components include the Light Emitting Diode (LED) 507 shown in FIG. 5, an audible output 509 such as a piezoelectric buzzer, electromechanical buzzer or alarm, or R-C time constant tone, siren, or beep, and a haptic output 511 such as a vibrator or eccentric shaft motor. The Pedicle Breach Detection Device of the present invention may have some or all of these output modes. In addition, the output modes may be user selectable to accommodate surgeon preference, operating room requirements, or the like. Additionally, the tactile surface 109 provides purely mechanical vibration to the surgeon independent of the electronic aspects of the device. The vibration and associated tactile feel results from probing the created tract or hole with the device of the present invention and the resulting transmission of that mechanical signal from the ball tip 103, through the shaft 103 and the shaft termination structure 107 to the tactile surface 109. The transmission of this mechanical signal may be considered an acoustic coupling of sorts.

While some embodiments of the present invention rely on the conversion of analog outputs from the accelerometer 111 into digital signals that in turn activate the various surgical alert components, further embodiments may also use analog accelerometer outputs with appropriate op-amp configurations to create vector summations or signal summations that are in turn used to drive a comparator with a threshold voltage and in turn output an analog signal or voltage that can either drive a surgical alert component directly or drive an amplifier or transistor to provide the appropriate gain necessary to meet the power requirements of the associated surgical alert component

It should also be noted that while an accelerometer may be used in a preferred embodiment, other sensors such as strain gauges may also be suitably used in the present invention. Strain gauges include, but are not limited to, resistive devices, semiconductor devices, piezoelectric devices and piezoresistors, fiber optic and optical devices, MEMs devices, and capacitive devices.

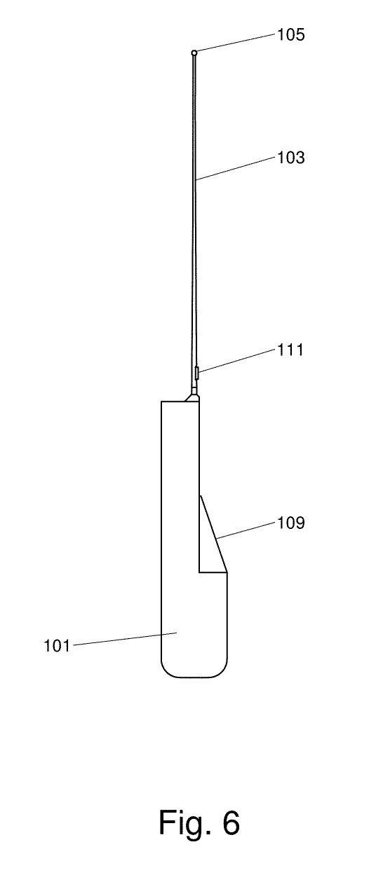

To gain a full understanding of the present invention and the various embodiments described, depicted and envisioned herein, FIG. 6 is an alternate side plan view of the pedicle breach detection device. While the tactile surface 109 is depicted as an angled form in relation to the axis of the surgical handle 101, placement, size and overall configuration of the tactile surface 109 may vary based on surgeon ergonomics as well as proper amplification or resonance of the mechanical signal from the ball tip 105 and shaft 103.

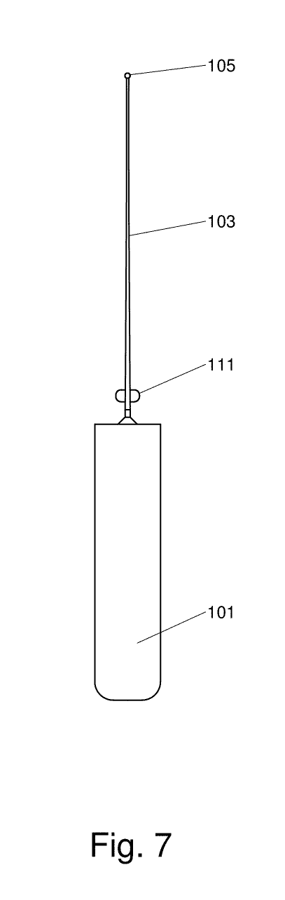

FIG. 7 is a bottom plan view of the pedicle breach detection device. The surgical handle 101 may take on various geometries depending on factors such as surgeon preference, procedural variations, and patient variations. The geometry of the surgical handle 101 depicted is to be considered, an example, and not a limitation.

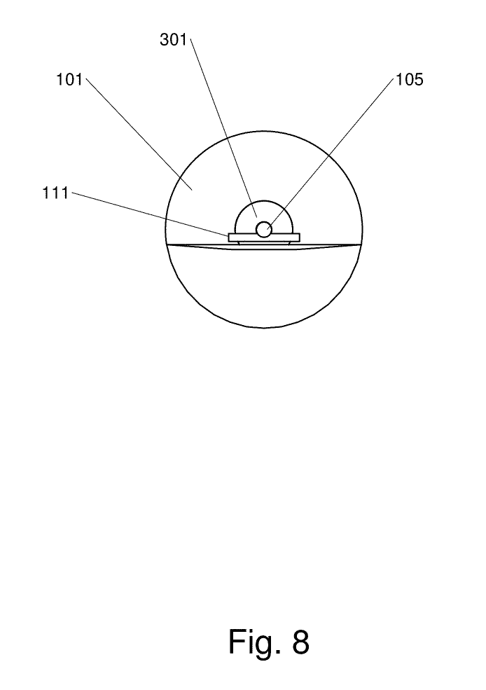

FIG. 8 is a surgical end view of the pedicle breach detection device. In other words, a view with the ball tip and shaft pointed toward the viewer, or out of the sheet.

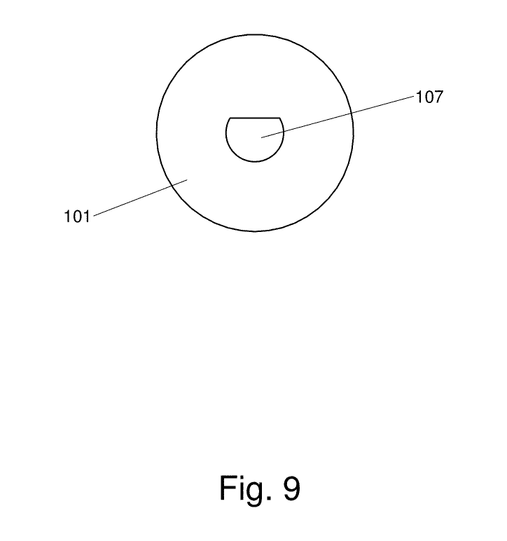

FIG. 9 is an operative end view of the pedicle breach detection device. In other words, the end of the surgical handle 101. While the shaft termination structure 107 can be seen extending through the handle structure itself, in some embodiments the shaft termination structure may be embedded or otherwise concealed within the surgical handle 101. In some embodiments of the present invention, existing ball tip probes may be used and inserted into the surgical handle with the coupling of the accelerometer to the shaft being removable or otherwise capable of being affixed to interchangeable or replaceable ball tip probes. The coupling of the accelerometer to the ball tip probe may employ springs, pads, screws, coils, or the like to facilitate the necessary mechanical attachment.

While the device of the present invention has been described so far as a breach detection device, the output signals from the accelerometer or sensor may contain further information that, with the appropriate signal processing and computational analysis, has value in other applications or provides additional information regarding the created surgical tract or hole that enhances and improves such surgical procedures.

FIG. 10 is a graph depicting output signals from the device and a vector summation signal of the three device output signals. In FIG. 10, outputs proportional to acceleration in each of the three axes can he seen in the top three traces, while the vector summation of each of these three outputs can be seen in the fourth, or bottom, trace.

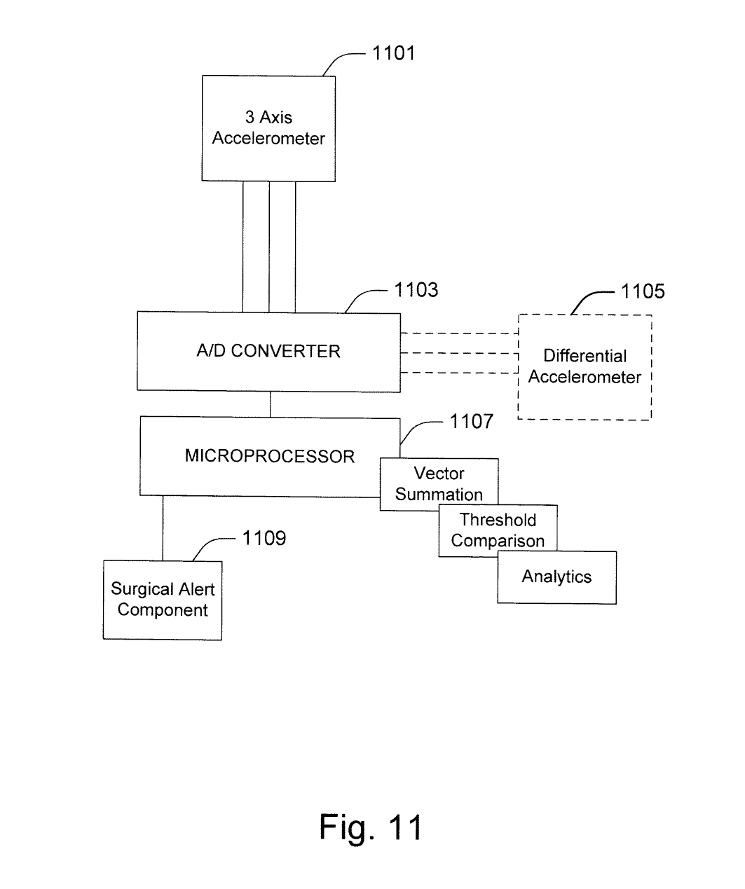

Turning now to FIG. 11, a block diagram depicting several of the major electronic components of the present invention can be seen. While most of these components have been previously described herein, a summary of their interconnections is presented for further clarity. An accelerometer 1101 such as a three axis accelerometer is mechanically coupled to the shaft of the surgical device of the present invention. This accelerometer 1101 provides axis specific outputs that are in turn connected to analog inputs of an analog to digital converter (A/D Converter) 1103. In some embodiments of the present invention, additional accelerometers are employed and appropriately input to the A/D converter 1103. In some alternate embodiments of the present invention, a differential accelerometer 1105 is used to pass unwanted signals (noise from vibration and the like) to the A/D converter 1103 of a further A/D converter that may be specific to the differential accelerometer 1105. The resulting digital signals from the A/D converter(s) are then used in or with a microprocessor(s) 1107 to perform method steps necessary for determining a surgical breach, as described herein. Such steps include, for example, vector summation of the axis specific signals that are digitized from the accelerometer outputs, a comparison of vector summations to a given or specified threshold, and analytics to improve future detection functionality through storage and processing of historical signals. If during the threshold comparison it has been determined that the threshold value(s) have been exceeded, the microprocessor 1107 will provide a signal or signals to activate the surgical alert components 1109 as previously described herein. While these major constituent components of the present invention provide the foundation for the functioning of the present invention, modifications, variations, additions and subtractions may occur, and are considered to be aspects or embodiments of the present invention as described and envisioned herein.

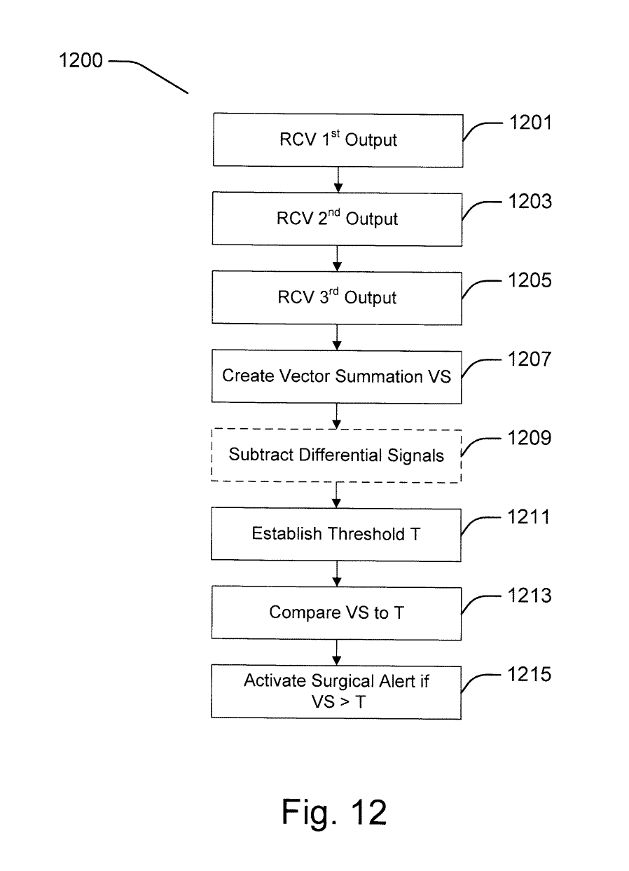

To provide an illustrative example of method steps of the present invention as implemented by the microprocessor(s), FIG. 12 is a flowchart depicting a computer program of the present invention.

An example of a computer program of the present invention would execute the steps of:

1201-receiving a first output from the analog to digital converter, the first output comprising a digital signal representative of a first axis analog signal. The first axis analog signal is from the three axis accelerometer where the first axis is the x-axis. The analog to digital converter(s) 501 (see FIG. 5) take this analog signal into the digital domain for subsequent processing.
1203-receiving a second output from the analog to digital converter, the second output comprising a digital signal representative of a second axis analog signal. The second axis analog signal is from the three axis accelerometer where the second axis is the x-axis. The analog to digital converter(s) 501 (see FIG. 5) take this analog signal into the digital domain for subsequent processing.
1205-receiving a third output from the analog to digital converter, the third output comprising a digital signal representative of a third axis analog signal. The third axis analog signal is from the three axis accelerometer where the third axis is the x-axis. The analog to digital converter(s) 501 (see FIG. 5) take this analog signal into the digital domain for subsequent processing.
1207-creating a vector summation value of the first output, the second output and the third output. This vector summation value may be a composite of the three outputs, or may, in some embodiments, be three discrete values from each of the three outputs. These discrete values may be average values over a specified time period, or may be maximum or minimum values, for example.
1209-<Optionally> a second accelerometer may be affixed to the operative end of the pedicle breach detection device, the output signals of the second accelerometer being subtracted from the output signals of the first accelerometer to remove unwanted noise from the system of the present invention.
1211-establishing a threshold value for activation of the surgical alert. This threshold value may be specified by the surgeon, or may be a threshold value that has been predetermined through an analysis of data from previous surgical procedures.
1213-comparing the vector summation value to the threshold value. This comparison may be a real-time, ongoing comparison for the duration of use of the Pedicle Breach Detection Device.
1215-activating the surgical alert if the vector summation value exceeds the threshold value. The activation of the surgical alert, be it a light, a tone or sound, or haptic feedback to the surgeon, is done in real-time while the slender shaft 103 and attached ball tip 105 are moved through the surgical pathway.

Data from the device of the present invention may also be used to create improved sensing and surgical feedback, and as such, storage of collected signals and transfer of those signals to an outside computing system is included in the present invention as described, claimed, and envisioned herein.

Use of the Pedicle Breach Detection Device is done in a way similar to that of a traditional ball tip probe where the surgeon moves the ball tip and shaft through a created tract, hole, surgical or anatomical feature in repeated circumferential and longitudinal motions. If the ball tip encounters a breach, an audible, visual, haptic and/or tactile signal is provided by the Pedicle Breach Detection Device. Such a signal may also vary in amplitude, duration and frequency based on the structure and extent of the breach encountered.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and the attached drawings.

What is claimed is;

1. A pedicle breach detection device comprising:
a slender shaft having a surgical end and an operative end;
a ball tip connected to the surgical end of the slender shaft;
a surgical handle connected to the operative end of the slender shaft;
an accelerometer mechanically coupled to the slender shaft: and
a surgical alert component configured to activate upon detection of a signal from the accelerometer that exceeds a threshold value.

2. The pedicle breach detection device of claim 1, wherein the surgical alert component is selected from the group consisting of an optical component, an audible component, a haptic component, and a tactile surface component.

3. The pedicle breach detection device of claim 1, further comprising an analog to digital converter electrically coupled to the accelerometer.

4. The pedicle breach detection device of claim 3, further comprising a microprocessor electrically coupled to the analog to digital converter.

5. The pedicle breach detection device of claim 1, further comprising a power source.

6. The pedicle breach detection device of claim 1, further comprising a shaft termination structure mechanically coupled to the operative end of the slender shaft and retained by the surgical handle.

7. The pedicle breach detection device of claim 1, further comprising a tactile surface.

8. The pedicle breach detection device of claim 1, wherein the slender shaft has an angle.

9. The pedicle breach detection device of claim 1, further comprising a transition taper between the slender shaft and the surgical handle.

10. The pedicle breach detection device of claim 1, further comprising a differential accelerometer mechanically coupled to the surgical handle.

11. A system for surgical breach detection comprising:
a slender shaft having a surgical end and an operative end;
a ball tip connected to the surgical end of the slender shaft;
a surgical handle connected to the operative end of the slender shaft;
an accelerometer mechanically coupled to the slender shaft;
a surgical alert component configured to activate upon detection of a signal from the accelerometer that exceeds a threshold value;
an analog to digital converter electrically coupled to an accelerometer output;
a computer program stored on computer readable media where the computer program executes the steps of;
receiving a first output from the analog to digital converter, the first output comprising a digital signal representative of a first axis analog signal;
receiving a second output from the analog to digital converter, the second output comprising a digital signal representative of a second axis analog signal;
receiving a third output from the analog to digital converter, the third output comprising a digital signal representative of a third axis analog signal;
creating a vector summation value of the first output, the second output and the third output;
establishing a threshold value for activation of the surgical alert;
comparing the vector summation value to the threshold value; and
activating the surgical alert if the vector summation value exceeds the threshold value.

12. A pedicle breach detection device comprising:
a slender shaft having a surgical end and an operative end;
a ball tip connected to the surgical end of the slender shaft;
a surgical handle connected to the operative end of the slender shaft;
a strain gauge mechanically coupled to the slender shaft; and
a surgical alert component configured to activate upon detection of a signal from the strain gauge that exceeds a threshold value.

13. The pedicle breach detection device of claim 12, wherein the surgical alert component is selected from the group consisting of an optical component, an audible component, a haptic component, and a tactile surface component.

14. The pedicle breach detection device of claim 12, further comprising an analog to digital converter electrically coupled to the accelerometer.

15. The pedicle breach detection device of claim 14, further comprising a microprocessor electrically coupled to the analog to digital converter.

16. The pedicle breach detection device of claim 12, further comprising a power source.

17. The pedicle breach detection device of claim 12, further comprising a shaft termination structure mechanically coupled to the operative end of the slender shaft and retained by the surgical handle.

18. The pedicle breach detection device of claim 12, further comprising a tactile surface.

19. The pedicle breach detection device of claim 12, wherein the slender shaft has an angle.

20. The pedicle breach detection device of claim 12, further comprising a transition taper between the slender shaft and the surgical handle.
